(19) United States
(12) Patent Application Publication
(10) Pub. No.: US 2021/0236042 A1
(43) Pub. Date: Aug. 5, 2021

(54) INTRACRANIAL ELECTRODE AND DELIVERY SYSTEM

(71) Applicant: Mayo Foundation for Medical Education and Research, Rochester, MN (US)

(72) Inventors: Jamie J. Van Gompel, Rochester, MN (US); Gregory A. Worrell, Rochester, MN (US); Mark A. Benscoter, Rochester, MN (US); Stephen T. Kuehn, Woodbury, MN (US); Seth A. Hara, Rochester, MN (US); Kendall D. Dennis, Rochester, MN (US); Joel L. Kuhlmann, Rochester, MN (US); Sanjeet S. Grewal, Jacksonville Beach, FL (US); Squire M. Stead, Rochester, MN (US); Stephan J. Goerss, Rochester, MN (US)

(21) Appl. No.: 17/050,885

(22) PCT Filed: May 1, 2019
(86) PCT No.: PCT/US2019/030224

§ 371 Date: Oct. 27, 2020

Related U.S. Application Data
(60) Provisional application No. 62/664,978, filed on May 1, 2018.

(51) Int. Cl. A61B 5/386; A61B 1/32; A61B 5/37; A61B 5/263; A61B 5/00; A61B 17/02; A61B 5/293

(52) U.S. Cl. CPC A61B 5/386; A61B 1/32; A61B 5/37; A61B 5/263; A61B 2562/168; A61B 17/0218; A61B 5/293; A61B 2560/063; A61B 5/4094

(57) ABSTRACT

A cortical access system for delivering a medical tool into an epidural and/or subdural space and onto a patient's brain tissue through a cranium opening comprises: a turret including a proximal end portion, a distal end portion, and a first channel extending from an entrance opening to an exit opening, the first channel configured to guide the medical tool from the entrance opening to the exit opening for positioning on the patients brain tissue. The medical tool may be an electrode or an endoscope.

### CROSS-REFERENCE TO RELATED APPLICATION

This application claims the benefit of U.S. Provisional Application 62/664,978, filed May 1, 2018, which is incorporated herein by reference in its entirety for all purposes.

### TECHNICAL FIELD

Various aspects of the instant disclosure relate to electrodes and electrode delivery tools. In some specific examples, the disclosure concerns intracranial electrodes and delivery systems.

### BACKGROUND

Epilepsy affects large patient populations. One third of epilepsy patients continue to have seizures despite medications. Surgery is the most effective treatment for medically resistant focal epilepsy, and can be a cure if the region generating seizures can be resected. Epilepsy surgery, however, is vastly under-utilized because localizing the focal brain region generating seizures is highly invasive, and associated with significant risk, patient discomfort, and expense. Currently, patients undergo electrode implantation for chronic intracranial EEG (iEEG) monitoring that extends over multiple days in order to capture and pinpoint the origin of seizures.

Epilepsy surgery can deliver a cure if the brain region generating seizures can be localized and removed. Surgical treatment for epilepsy is based on the concept that seizures begin in a focal region, the seizure onset zone (SOZ), and then propagate to susceptible tissue. For seizure freedom the SOZ and surrounding epileptogenic zone (EZ) must be removed. In current practice, early seizure propagation and active inter-ictal spiking determine the EZ. In addition, if the SOZ is accurately localized, electrodes can be implanted permanently for therapeutic electrical brain stimulation.

Intracranial EEG (iEEG) is the gold standard for localizing the SOZ and EZ. Patients have electrodes implanted via a large craniotomy. The evaluation requires multiple days of iEEG to capture spontaneous seizures, and define the SOZ and surrounding EZ. The long duration of iEEG monitoring is driven by the need to record seizures (unpredictable events requiring days to capture). Patients are hospitalized in the Neuro ICU at considerable cost. They experience significant discomfort and risk of morbidities.

There remains a continuing need for improved electrodes, including but not limited to intracranial EEG electrodes, delivery tools and associated methods. In particular, there is a need for such electrodes, tools and methods that can improve outcomes. Technologies of these types that can efficiently decrease invasiveness and morbidity would be especially desirable.

### SUMMARY OF THE INVENTION

In various embodiments, a cortical access system for delivery of one or more electrodes into an epidural and/or subdural space and onto a patient's brain tissue through a cranium opening comprises: a turret including a proximal end portion, a distal end portion, and a first channel extending from an entrance opening to an exit opening, the first channel configured to guide the one or more electrodes from the entrance opening to the exit opening for positioning on the patient's brain tissue.

In some examples, the distal end portion includes a periphery; and the exit opening of the first channel is on the periphery of the distal end portion, and the channel is configured to guide the one or more electrodes onto the patient's brain tissue below the cranium.

In some examples, the turret further comprises a second channel configured for the one or more electrodes to be released from the turret.

In some examples, the second channel is narrower than the first channel such that only a portion of each of the one or more electrodes is releasable via the second channel.

In some examples, the first channel is curved or sloped to form a ramp for guiding the one or more electrodes out of the exit opening generally horizontally between the patient's brain tissue and cranium.

In some examples, the turret comprises a turret frame and a turret insert removably mated to the turret frame, and wherein the turret insert defines the first channel.

In some examples, the cortical access system further comprises a mounting plate configured to be secured to the patient's cranium at the cranial opening via securing elements and to rotatably receive the turret.

In some examples, the cortical access system further comprises a retaining ring configured to be coupled to the mounting plate such that the turret is rotatable while translational motion of the turret is limited.

In some examples, the turret is adjustable for different skull thicknesses.

In some examples, the turret further comprises an endoscope channel configured to receive an endoscope for imaging of an electrode being delivered by the system.

In some examples, the turret further comprises a force sensor configured to monitor a force exerted onto the patient's brain tissue.

In some embodiments, an electrode comprises: a head having a proximal end portion and a distal end portion wider than the proximal end portion; a tail extending from the proximal end portion of the head; and one or more electrode contacts disposed on the head; wherein the electrode is flexible and configured to be inserted through a cortical access system to access a patient's brain tissue.

In some examples, the electrode further comprises a bumper at the distal end portion of the head.

In some examples, the bumper is radiopaque.

In some examples, the bumper is configured to releasably receive a guiding tool.

In some examples, manipulation of the guiding tool causes movement of the electrode.

In some examples, the tail is configured to be releasably coupled to the guiding tool via a clamp.

In some examples, the electrode further comprises a tab at a proximal end of the tail.

In some examples, the electrode comprises a biocompatible dielectric substrate, a conductive layer coupled to the substrate, and a biocompatible dielectric top layer coupled to the conductive layer.

In some examples, the biocompatible dielectric substrate and/or the biocompatible dielectric top layer comprises at least one of polyimide, Parylene-C, and silicone.

In some examples, the conductive layer comprises at least one of platinum, titanium-platinum, gold, copper, and tin.

In some examples, the head is configured to be movable through a first channel of the cortical access system and not movable through a second channel of the cortical access system.

In some examples, the tail is configured to be movable through the second channel of the cortical access system.

In some examples, the tail is configured to be manipulated such that the electrode is releasable from a turret of the cortical access system to allow another electrode to be inserted through the cortical access system.

In some examples, the body is generally wedge-shaped such that a plurality of the electrodes is circumferentially distributable on the patient's brain tissue.

In some examples, the electrode further comprises one or more fluid chambers disposed at least at the head.

In some examples, each fluid chamber is fluidically connected to a fill tube at a proximal end of the electrode.

In some examples, the fill tube is configured to transport fluid in and out of each fluid chamber to change fluid quantities in the fluid chamber.

In some examples, the one or more fluid chambers are configured to transition the electrode between an initial state and a positive state in response to a change in fluid quantity in the one or more fluid chambers.

In some examples, the electrode has a variable stiffness corresponding to a fluid quantity in the one or more fluid chambers.

In some examples, the electrode is configured to move in response to a sequential change in fluid quantities in the one or more chambers.

In some examples, the electrode is configured to transition between a delivery state at which the electrode has a first width, and a deployed state at which the electrode has a second width greater than the first width, in response to change in fluid quantities in the one or more chambers.

In some embodiments, a method for deploying one or more electrodes of an intracranial apparatus including a cortical access system having a turret with a first channel includes: deploying the cortical access system at a patient's cranial opening; inserting an electrode through the patient's cranial opening via the first channel to access the patient's brain tissue; and releasing the electrode from the turret such that the turret is rotatable independently of the released electrode.

In some examples, the cortical access system further includes a mounting plate and a retaining ring, wherein deploying the cortical access system comprises: coupling the mounting plate to a patient's cranium at the cranial opening via securing elements; coupling the turret to the mounting plate; and coupling the retaining ring to the mounting plate such that the turret is rotatable.

In some examples, the turret includes a turret frame and a turret insert, and wherein coupling the turret to the mounting plate includes: coupling the turret frame to the mounting plate; and coupling the turret insert to the turret frame.

In some examples, inserting the electrode comprises guiding the electrode's head out of the first channel to a location between the patient's cranium and brain tissue.

In some examples, guiding the electrode's head comprises: coupling a guiding tool to the electrode head; and manipulating the guiding tool to guide the electrode.

In some examples, coupling the guiding tool comprising coupling the guiding tool to a bumper on the electrode's head.

In some examples, guiding the electrode's head further comprises verifying the placement of the electrode via visualizing the location of the bumper.

In some examples, guiding the electrode's head further comprising coupling the electrode and the guiding tool via a clip.

In some examples, inserting the electrode comprises advancing the electrode such that the clip comes into contact with the turret.

In some examples, releasing the electrode comprises removing the clip such that the electrode's tail is manipulatable independently from the guiding tool.

In some examples, releasing the electrode comprises removing the guiding tool from the electrode.

In some examples, the electrode is configured to be released such that the turret is rotatable independently from the electrode.

In some examples, the method for deploying one or more electrodes of an intracranial apparatus further comprises: rotating the turret such that another of the one or more electrodes can be inserted; and inserting another of the one or more electrodes.

In some examples, the method for deploying one or more electrodes of an intracranial apparatus further comprises: repeating rotating the turret and inserting another of the one or more electrodes such that the one or more electrodes are deployed circumferentially.

In some examples, the one or more electrodes are deployed circumferentially to cover a substantial area within a circle.

In some examples, the method for deploying one or more electrodes of an intracranial apparatus further comprises: inserting an endoscope through the patient's cranial opening via the cortical access system and using the endoscope to visualize the electrode.

In some examples, releasing the electrode from the turret comprises releasing the electrode through a second channel in the turret that is connected to the first channel.

In some embodiments, a cortical access system for delivering a medical tool into an epidural and/or subdural space and onto a patient's brain tissue through a cranium opening comprises: a turret including a proximal end portion, a distal end portion, and a first channel extending from an entrance opening to an exit opening, the first channel configured to guide the medical tool from the entrance opening to the exit opening for positioning on the patient's brain tissue.

In some examples, the distal end portion includes a periphery; and the exit opening of the first channel is on the periphery of the distal end portion, and the channel is configured to guide the medical tool onto the patient's brain tissue below the cranium.

In some examples, the first channel is curved or sloped to form a ramp for guiding the medical tool out of the exit opening generally horizontally between the patient's brain tissue and cranium.

In some examples, the turret comprises a turret frame and a turret guide removably mated to the turret frame, and wherein the turret guide defines the first channel.

In some examples, the cortical access system further comprises a turret base configured to be secured to the patient's cranium at the cranial opening via a securing means and to rotatably receive the turret.

In some examples, the cortical access system further comprises a turret lock configured to be coupled to the turret base such that the turret is rotatable while translational motion of the turret is limited.

In some examples, the cortical access system further comprises a guide clamp configured to be coupled to the turret to help secure the medical tool received in the first channel.

In some examples, the cortical access system further comprises a clamp lock configured to secure the guide clamp to the turret guide.

In some examples, the medical tool is an electrode or an endoscope.

### BRIEF DESCRIPTION OF THE DRAWINGS

FIG. 1 shows an intracranial apparatus, according to some examples.

FIG. 2 shows a cortical access system of the intracranial apparatus of FIG. 1, according to some examples.

FIG. 3 shows an exploded view of the cortical access system of FIG. 2, according to some examples.

FIG. 4 shows a cross-sectional view of the cortical access system of FIG. 2 (section A-A) coupled to a patient's skull, according to some examples.

FIG. 5 shows turret frames configured for use in different cranium thicknesses, according to some examples.

FIG. 6 shows an electrode of the intracranial apparatus of FIG. 1, according to some examples.

FIG. 7 shows the intracranial apparatus of FIG. 1 before insertion of an electrode into a cortical access system, according to some examples.

FIG. 8 shows the intracranial apparatus of FIG. 1 during insertion of an electrode into a cortical access system, according to some examples.

FIG. 9 shows the intracranial apparatus of FIG. 1 after the insertion of an electrode into a cortical access system, according to some examples.

FIG. 10 shows a cross-sectional view of the intracranial apparatus of FIG. 9 (section B-B), according to some examples.

FIG. 11 shows the intracranial apparatus of FIG. 1 with an electrode inserted into a cortical access system and deployed, according to some examples.

FIG. 12 shows the intracranial apparatus of FIG. 11 with the guiding tool removed, and the turret frame and turret insert positioned for electrode deployment in a different location, according to some examples.

FIG. 13 shows one or more electrodes deployed and without guiding tools, according to some examples.

FIG. 14 shows an electrode of the intracranial apparatus of FIG. 1, according to some examples.

FIG. 15A shows the electrode of FIG. 14 in a rolled-up state, viewing from the front, according to some examples.

FIG. 15B shows the electrode of FIG. 14 in a rolled-up state, viewing from the proximal end, according to some examples.

FIG. 16 depicts an illustrative method for deploying one or more electrodes of an intracranial apparatus, according to some examples.

FIG. 17 depicts an illustrative method for deploying a cortical access system, according to some examples.

FIG. 18 depicts an illustrative method for inserting an electrode via a first channel, according to some examples.

FIG. 19 depicts an illustrative method for releasing an electrode from a turret insert via a second channel, according to some examples.

FIG. 20 shows a cortical access system, according to some examples.

FIG. 21 shows the cortical access system of FIG. 20 in an exploded view, according to some examples.

While the disclosure is amenable to various modifications and alternative forms, specific embodiments have been shown by way of example in the drawings and are described in detail below. The disclosure, however, is not limited to the particular embodiments described. On the contrary, the disclosure is intended to cover all modifications, equivalents, and alternatives falling within the scope of the disclosure as defined by the appended claims.

### DETAILED DESCRIPTION

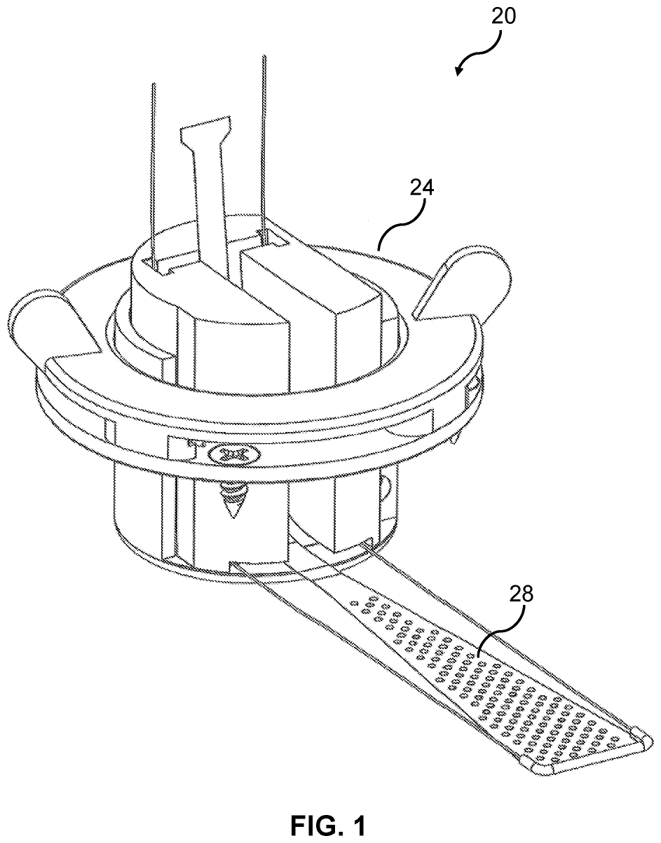

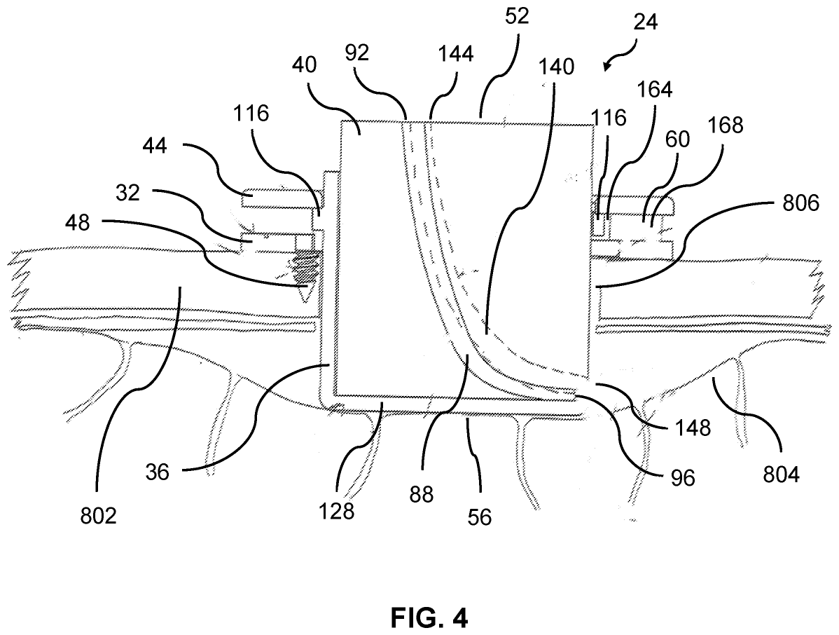

FIG. 1 shows an intracranial apparatus 20, according to some examples. The intracranial apparatus 20 includes a cortical access system 24 and an electrode 28 configured to be inserted through the cortical access system 24 and into a patient's skull. The cortical access system 24 is configured to be mounted to the patient's skull, particularly to the patient's cranium 802 (see FIG. 4) in some examples to aid the insertion of the electrode 28 to a target intracranial location, such as on the patient's brain tissue 804 (see FIG. 4).

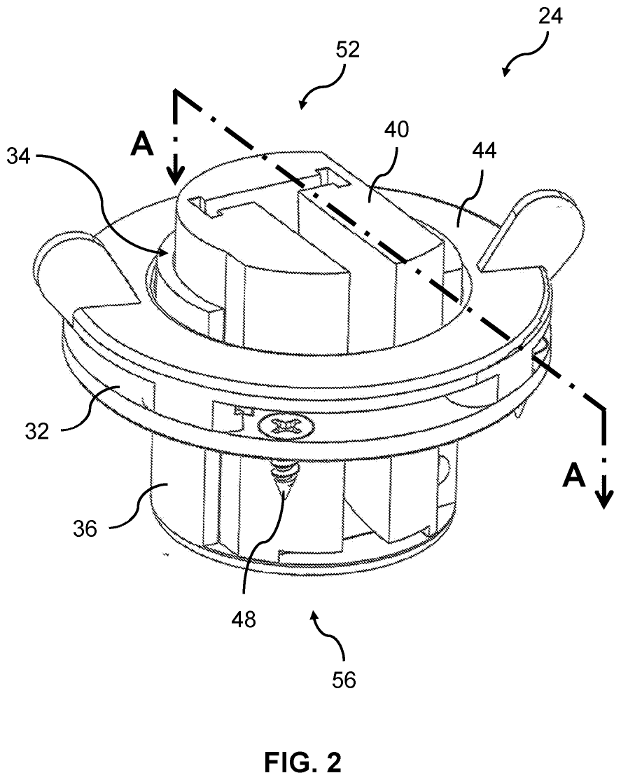

FIG. 2 shows the cortical access system 24 of the intracranial apparatus 20 of FIG. 1, according to some examples. The cortical access system 24 includes a mounting plate 32 configured to be mounted to the patient's skull and to receive a turret 34. The turret 34 may include a turret frame 36 and a turret insert 40. The turret frame 36 is configured to receive, for example, removably receive a turret insert 40. The turret frame 36 and/or the turret insert 40 may be cylindrical and may be separate components or a single component. The cortical access system 24 further includes a retaining ring 44 configured to be coupled to the mounting plate 32 to limit translational motion but allow rotational motion of the turret frame 36 and/or turret insert 40 with respect to the mounting plate. The cortical access system 24 further includes securing elements 48 (e.g., screws, bolts, clips, and/or O-rings) to couple, such as fixedly couple the mounting plate 32 to the patient's skull. The cortical access system 24 has an upper or proximal end 52 and a lower or distal end 56. The proximal end 52 may be extracranial and the distal end 56 may be intracranial when the cortical access system 24 is mounted to the patient's skull.

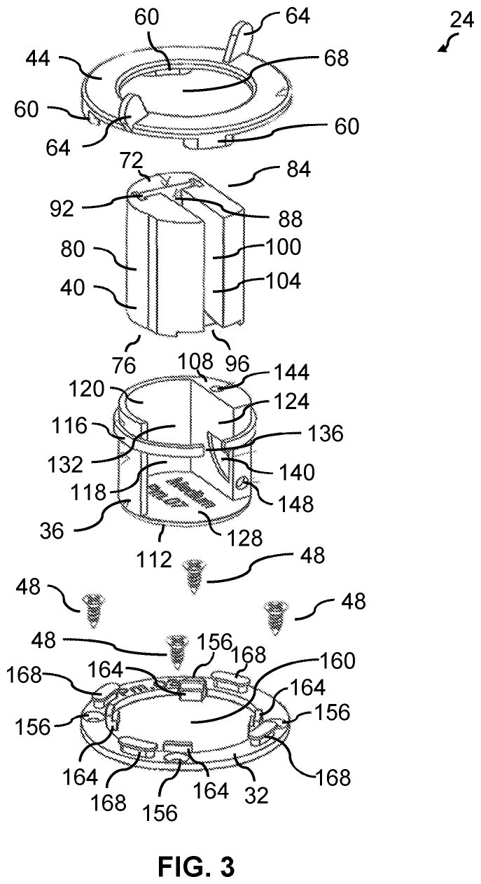

FIG. 3 shows an exploded view of the cortical access system 24 of FIG. 2, according to some examples. The retaining ring 44 includes one or more coupling members 60 (e.g., clips, magnets, snap-fit connectors, and/or tight-fit connectors) configured to be coupled to the mounting plate 32. The retaining ring 44 may further include one or more leverage structures 64 configured to help a user to secure (e.g., via rotation motion and/or translational motion) the retaining ring 44 onto the mounting plate 32. The retaining ring 44 may engage the mounting plate 32 to enable the turret frame 36 to rotate with respect to the mounting plate. The retaining ring 44 may further include an opening 68 configured to receive the turret frame 36 and/or the turret insert 40. Although shown as being circular in the illustrated embodiments, the retaining ring 44 may take other shapes in other embodiments.

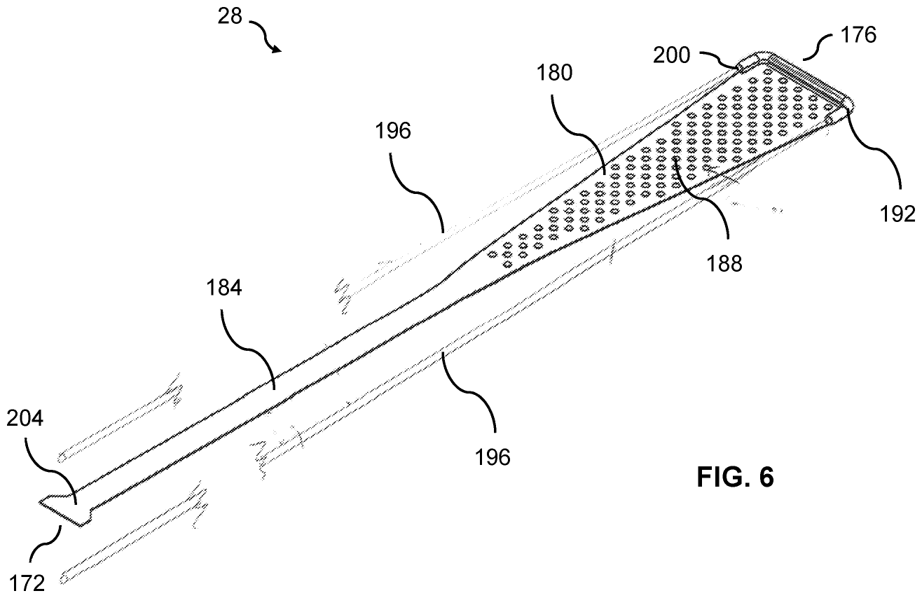

As shown, the turret insert 40 has a proximal end 72 or an upper portion having a periphery, a distal end 76 or a lower portion including a periphery, and a first surface 80 (e.g., a curved surface) configured to be coupled or mated with the turret frame 36. The turret insert 40 may further include a second surface 84 (e.g., an oblique surface) configured to be coupled or mated with the turret frame 36. One or both of the surfaces 80, 84 may help position the turret insert 40 in the turret frame 36 in a target orientation (e.g., an orientation where an electrode 28 (see FIG. 6) may be inserted through the turret frame and the turret insert). Additionally or alternatively, one or both of the surfaces 80, 84 may help couple the turret insert 40 to the turret frame 36 such that the turret insert and the turret frame rotate together.

The turret insert 40 further has or defines a delivery or first channel 88 extending from an entrance opening 92 at the proximal end 72 to a side exit or exit opening 96 near the distal end 76 (e.g., on the periphery of the lower portion). The exit opening 96 may be at least partially directed horizontally (e.g., parallelly to the base 128 of the turret frame 36). The first channel 88 may be configured to guide or direct an electrode 28 (see FIG. 6) or any other tools or components from outside of the skull to inside of the skull (e.g., by entering from the entrance opening 92 and extending out from the exit opening 96). The first channel 88 and/or the entrance opening 92, and/or the exit opening 96 may be wide and rectangular as illustrated or take up other shapes to allow other tools (e.g., rolled electrodes, grasping tools, cutting tools, and suction tools) to be inserted. The first channel 88 may be curved or slanted (see FIG. 4) to form a guide ramp such that an electrode 28 (see FIG. 6) may be inserted or guided (e.g., from the entrance opening 92 at the proximal end 72 and emerge out from the horizontally directing exit opening 96) to between the patient's skull and brain tissue (e.g., to be near or in contact with or on the patient's brain tissue) by being extended out from the exit opening 96 substantially horizontally or parallelly to the mounting plate 32, the retaining ring 44, and/or a base 128 of the turret frame 36. The turret insert 40 further has or defines a removal or second channel 100 extending from the proximal end 72 to the distal end 76 and coupled to the first channel 88. The second channel 100 may be an open channel having a removal or side opening 104 also extending from the proximal end 72 to the distal end 76. The second channel 100 may be narrower than the first channel 88 such that a portion (e.g., the tail portion 184) of an electrode 28 (see FIG. 6) may be moved through the second channel to decouple the electrode from the turret insert 40.

As shown, the turret frame 36 has a proximal end 108 or an upper portion having a periphery, a distal end 112 or a lower portion having a periphery, and a retaining structure 116 (e.g., a rim, a ring, and/or a groove) configured to be coupled to, such as rotatably coupled to the mounting plate 32 and/or retaining ring 44. The turret frame 36 further has an insert opening 118 configured to receive the turret insert 40 and a first surface 120 (e.g., a curved surface) configured to be coupled to the turret insert 40 (e.g., to the first surface 80). The turret frame 36 may further have a second surface 124 (e.g., an oblique surface) configured to be coupled with the turret insert 40 (e.g., to the second surface 84). One or both of the surfaces 120, 124 may help position the turret insert 40 in the turret frame 36 in a target orientation (e.g., an orientation where an electrode 28 (see FIG. 6) may be inserted through the turret frame and the turret insert). Additionally or alternatively, one or both of the surfaces 120, 124 may help couple the turret insert 40 to the turret frame 36 such that the turret insert and the turret frame rotate together. The turret frame 36 may further includes a base 128 at the distal end 112 configured to be coupled to the distal end 76 of the turret insert 40 to help limit relative translational motion between the turret insert and the turret frame.

The turret frame 36 further defines or has a first side opening 132 configured to be aligned to or adjacent the exit opening 96 of the turret insert 40 such that an electrode 28 (see FIG. 6) may extend out from the exit opening (e.g., to reach an intracranial target location). The first side opening 132 may extend from the proximal end 108 to the distal end 112 or the base 128 and be at least as wide as the exit opening 96 of the turret insert 40. The turret frame 36 further defines or has a second side opening 136 configured to be aligned or oriented with the side opening 104 of the second channel 100 of the turret insert 40 such that only a portion of an electrode 28 (see FIG. 6) may be moved through the second side opening 136. The second side opening 136 may be defined by the retaining structure 116 (e.g., a split ring) and may be at least as wide as the side opening 104 of the second channel 100 of the turret insert 40.

The turret frame 36 may further include a tool channel (e.g., an endoscope channel 140) connecting a tool entrance opening (e.g., an endoscope entrance opening 144) and a tool exit opening (e.g., an endoscope exit opening 148). The tool channel or endoscope channel 140 may be curved or slanted (see FIG. 4) such that a tool (e.g., an endoscope 152) (see FIGS. 6-8) may be guided or inserted between the patient's skull and brain tissue by being extended out from the exit opening substantially parallelly to the mounting plate 32, the retaining ring 44, and/or the base 128 of the turret frame 36. For example, the endoscope exit opening 148 may be at least partially directed horizontally (e.g., parallelly to the base 128 of the turret frame 36). The endoscope exit opening 148 may be adjacent to the electrode exit opening 96 of the turret insert 40 and oriented to enable imaging of an electrode being delivered.

As shown, the mounting plate 32 may have or define one or more securement openings 156 configured to receive the securing elements 48 (e.g., screws or pins) to secure the mounting plate to the patient's skull. The mounting plate 32 further includes an opening 160 (e.g., a central opening) configured to receive, such as rotatably receive the turret frame 36 and/or the turret insert 40. For example, the mounting plate 32 may include one or more retaining structures 164 (e.g., stepped supports) configured to be coupled, such as rotatably coupled with the retaining structure 116 of the turret frame 36. The mounting plate 32 may further include one or more coupling members 168 (e.g., clips, magnets, snap-fit connectors, and/or tight-fit connectors) configured to cooperate with the coupling members 60 of the retaining ring 44 to limit translational motion but allow rotational motion of the turret frame 36 and/or turret insert 40 relative to the mounting plate.

FIG. 4 shows a cross-sectional view of the cortical access system 24 of FIG. 2 (section A-A) coupled to a patient's skull, according to some examples. The cortical access system 24 is configured to be received in a craniotomy or cranial opening 806 of the patient's skull or cranium 802 such that tools or components (e.g., electrodes 28 of FIG. 6) of the intracranial apparatus 20 may be positioned near or on the patient's brain tissue 804. The mounting plate 32 is mounted, coupled, secured, or attached to the cranium 802 near the cranial opening by the securing elements 48 (e.g., screws). The opening 160 of the mounting plate 32 may be substantially the same size as the cranial opening 806, such as within 20% difference in area, such as within 10% difference in area, such as within 50% difference in area. The turret frame 36 and the turret insert 40 received in the turret frame may be rotatably coupled to the mounting plate 32, for example, by rotatably coupling the retaining structure 116 of the turret frame to the retaining structure 164 of the mounting plate. The retaining ring 44 may then be coupled to the mounting plate 32 by coupling the coupling members 60 of the retaining ring to the coupling members 168 of the mounting plate 32.

As shown, when the cortical access system 24 is deployed, the proximal end 52 may be extracranial and the distal end may be intracranial such that a user may manipulate tools and/or components in a patient's skull (e.g., electrodes 28 of FIG. 6 and endoscopes 152) from outside of the patient's skull. The base 128 or the distal end 112 of the turret frame 36 may be positioned significantly near or in contact with the brain tissue 804 at the cranial opening 806 when the cortical access system 24 is deployed. The cortical access system 24 may include curved surfaces to aid its coupling to the patient's skull. The first channel 88 of the turret frame 36 may be curved such that an electrode 28 (see FIG. 6) or other components may be inserted from the entrance opening 92 and guided out of the exit opening 96 substantially parallel to the base 128 of the turret frame 36 to be positioned near or in contact with the brain tissue 804. In the illustrated example, the exit opening 96 is on the side or peripheral surface of the turret insert 40 to facilitate the delivery of the tools into spaces between the patient's skull or cranium 802 and brain tissue 804. Similarly, a tool such as an endoscope 152 may be inserted through a channel such as the endoscope channel 140 (e.g., a curved channel) from the entrance opening 92 and out of the exit opening 96 substantially parallel to the base 128 of the turret frame 36 to be positioned near or in contact with the brain tissue 804. In embodiments, the channel 140 is located and configured to enable tools such as an endoscope delivered through to cooperate with the delivery of electrodes (e.g., to allow imaging of the electrodes).

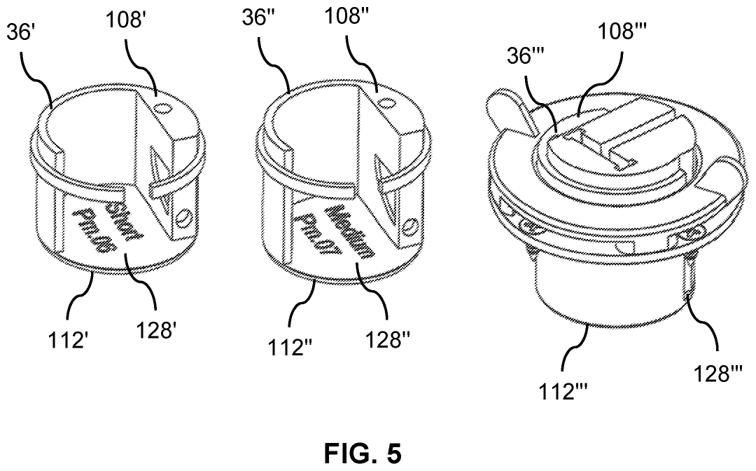

FIG. 5 shows turret frames 36′, 36″, and 36′″ configured for use in different cranium thicknesses (e.g., 1-20 mm), according to some examples. It is to be understood that turret frames 36′, 36″, and 36′″ may be substantially similar to turret frame 36 and may include features and/or elements of turret frame 36. For example, turret frames 36′, 36″, and 36′″ include proximal ends 108′, 108″, and 108′″, distal ends 112′, 112″, and 112′″, and bases 128′, 128″, and 128′″, respectively. Each of the turret frames 36′, 36″, and 36′″ defines a length or depth (i.e., distance from the proximal end to the distal end or distance from the proximal end to the base) which may be designed, modified, or adjusted for use in different cranium thicknesses. For example, each of a plurality of turret frames may have a length or depth appropriate for use with a particular skull thickness or a range of thickness. Additionally or alternatively, the length or depth of a turret frame 36 may be adjustable to be appropriate for use with a range of skull thickness. A pressure or force sensor may be positioned at the base to help monitor and/or limit force exerted onto the brain tissue to help limit brain tissue damage.

FIG. 6 shows the electrode 28 of the intracranial apparatus 20 of FIG. 1, according to some examples. The electrode 28 includes a body portion, a proximal end 172, a distal end 176, a first and second side portions, a head portion 180 near the distal end, and a tail portion 184 near the proximal end. The proximal end may extend from the proximal end portion of the body portion. The body portion may define an inclusive angle between 5° and 45°. The electrode may further include a non-traumatic tip on the distal end or distal end portion of the body. The electrode 28 may be wider at the distal end 176 or the distal end portion than at the proximal end 172 or the proximal end portion. The first and second side portions may be generally straight. The body portion may be elongated, and in at least some embodiments has a length that is at least two times, such as at least three times as long as the width of the distal end portion. The electrode 28 may include one or more electrode contacts 188 or conductive contact pads disposed at least on or in the head portion 180 of the electrode (e.g., on the body portion). A conductive trace may extend from each of the one or more electrode contacts 188. The electrode 28 may further include a bumper 192 or a delivery tool attachment structure at the distal end or on the body portion configured to help the insertion (e.g., by providing additional stiffness) of the electrode through the cortical access system 24 (see FIG. 1) and/or intracranial positioning. The bumper 192 may be radiopaque to allow verification of the placement of the electrode 28 (e.g., via fluoroscopy). The electrode 28 (e.g., the body portion) may be configured to releasably receive (e.g., near the distal end 176 and/or the bumper 192) a delivery or guiding tool 196 (e.g., a guide wire) to help the insertion of the electrode. For example, the electrode 28 near the distal end 176 and/or the bumper 192 may define a distal pocket or channel 200 configured for the guiding tool 196 to extend through.

The delivery tool attachment structure may include a distal attachment structure on the distal end portion of the body portion. The delivery tool distal attachment structure may include a radiused member (e.g., a tubular bumper 192 having a channel 200) configured for the delivery tool (e.g., the guide wire) to extend through. The bumper 192 may provide rigidity along the distal (i.e., leading) edge and side edges of an electrode (e.g., electrode 28) such that force exerted (e.g., by a user) to the delivery tool may allow force to be transferred to the distal edge while inhibiting folding, creasing, and/or damaging of the electrode. The delivery tool attachment structure may include a side attachment structure on one or both of the first or second side portions of the body portion. The delivery tool side attachment structure may include a tubular member. The delivery tool may be configured for attachment to the delivery tool side attachment structure and include a wire configured to extend through the tubular member of the side attachment structure. An electrode (e.g., electrode 28) may generally be deployed by applying a force to its distal end such that the electrode is not urged to buckle.

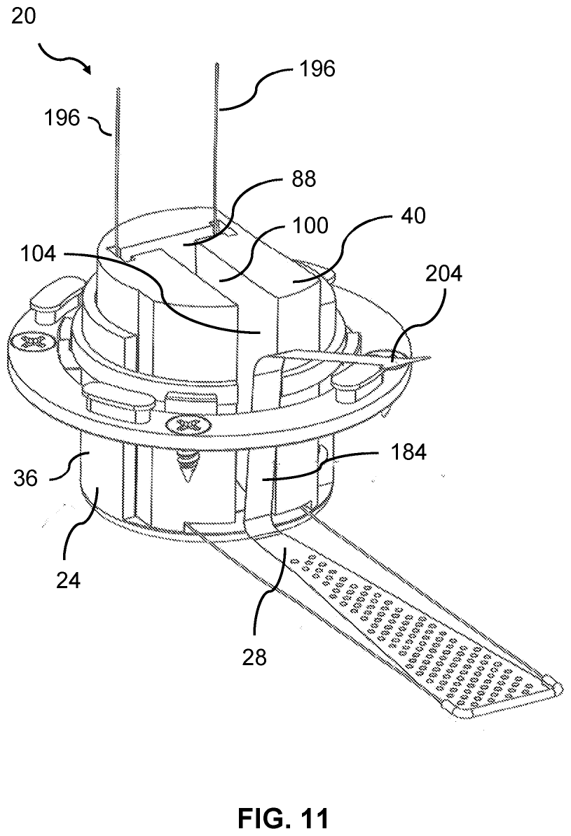

The electrode 28 may further include a tab 204 at the proximal end 172 and configured to be manipulated by a user, for example, for bending the tail portion 184 of the electrode (see FIG. 11) and/or for mounting an electrical connector.

The electrode 28 may be a flexible thin film laminate including a biocompatible dielectric substrate (e.g., polyimide, Parylene-C, and/or silicone), a conductive layer coupled to the substrate and forming electrical connections between the one or more electrode contacts 188, and a dielectric top layer having openings at the contacts and the connection pad. The conductive layer may include conductive material such as platinum, titanium-platinum, gold, copper, and/or tin. The dielectric top layer also includes biocompatible material such as polyimide, Parylene-C, and/or silicone. Additionally or alternatively, the electrode contacts 188 may be formed via electroplating, physical vapor deposition, chemical vapor deposition, photolithography, soft lithography, and/or ink-based printing. The electrode 28 may be 5-100 microns thick.

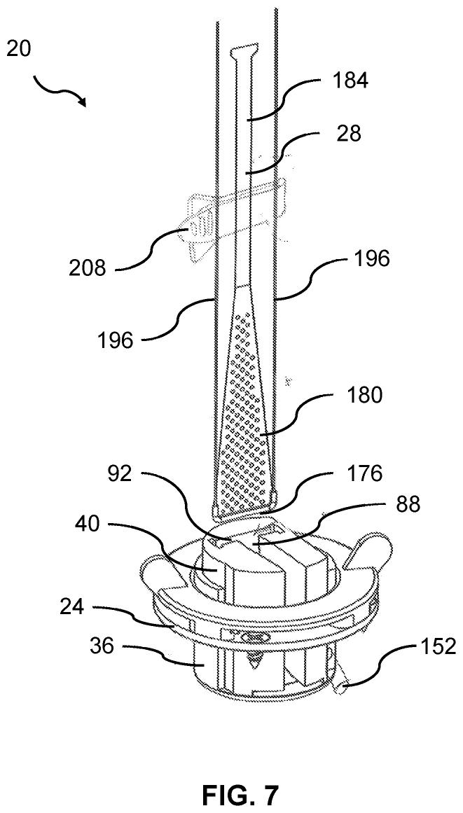

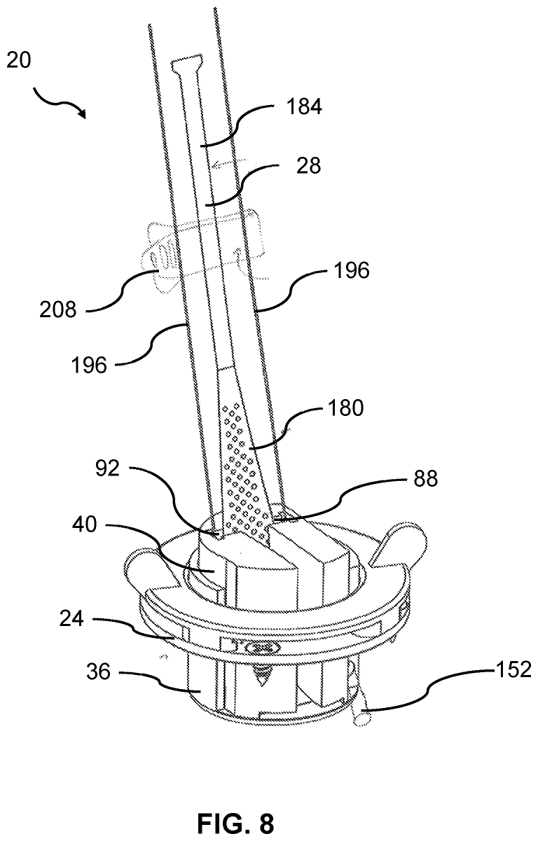

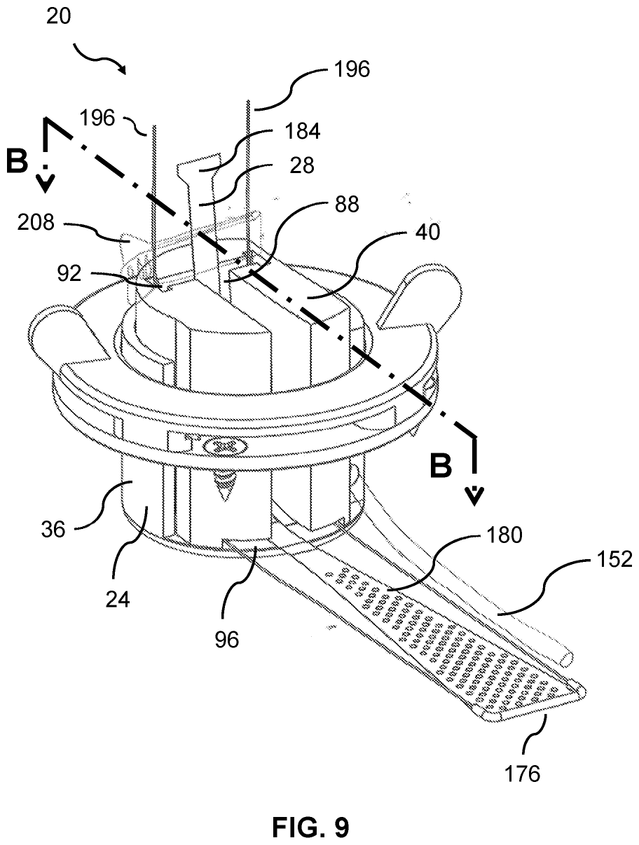

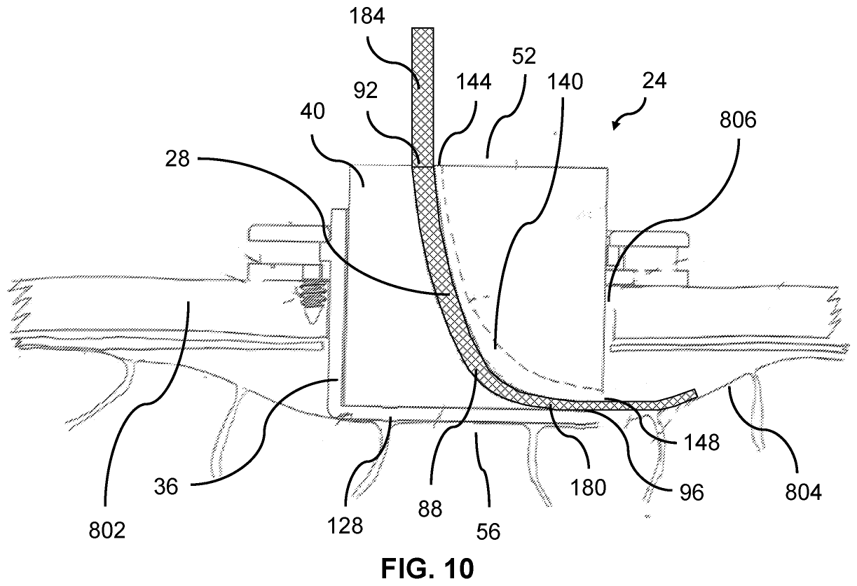

FIGS. 6-8 show the intracranial apparatus 20 of FIG. 1 before (FIG. 7), during (FIG. 8), and after (FIG. 9) the insertion of the electrode 28 into the cortical access system 24, according to some examples. FIG. 10 shows a cross-sectional view of the intracranial apparatus 20 of FIG. 9 (section B-B), according to some examples. The electrode 28 is inserted into the cortical access system 24 by guiding or feeding the distal end 176 through the entrance opening 92 of the first channel 88 of the turret insert 40 optionally with the help of the guiding tool 196. Following the distal end 176, the head portion 180 and then the tail portion 184 of the electrode 28 is inserted, guided, or fed through the cortical access system 24 to exit from the exit opening 96 of the turret insert 40. To help stabilize the guiding tool 196 and/or to limit advancement of the electrode insertion, a coupling member or a clamp or a clip 208 may be used to couple the guiding tool 196 with the electrode 28 (e.g., the tail portion 184 of the electrode). For example, the guiding tool 196 may be coupled to the electrode 28 by the clip 208 such that the electrode may be manipulated via manipulation of the guiding tool (e.g., by a user). The electrode may be inserted or advanced beyond the exit opening 96 up to when the clip comes into contact with the turret frame 36 or turret insert 40. As shown, the electrode 28 may extend out of the exit opening 96 substantially parallelly to the base 128 of the turret frame 36 to access between the patient's cranium 802 and brain tissue 804 to be near or in contact with the brain tissue.

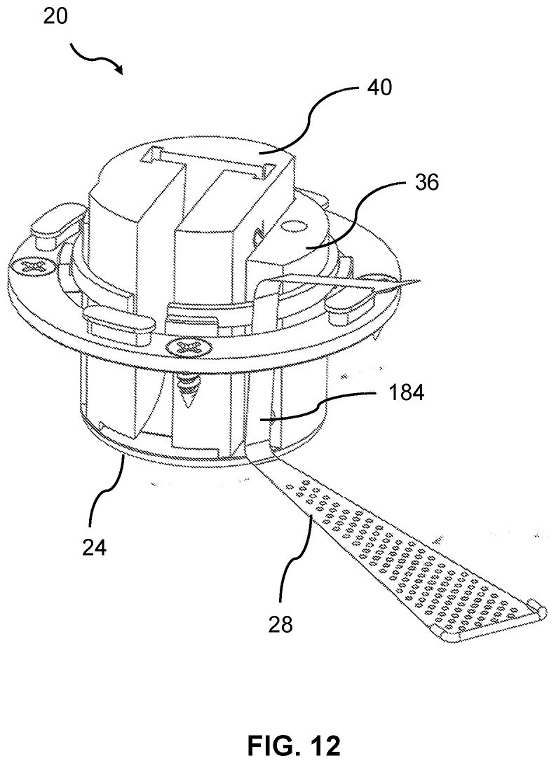

FIG. 11 shows the intracranial apparatus 20 of FIG. 1 with the electrode 28 inserted into the cortical access system 24 and deployed, according to some examples. FIG. 12 shows the intracranial apparatus of FIG. 11 with the guiding tool 196 removed, according to some examples. After the electrode 28 is inserted (FIG. 9), the clip 208 may be removed to allow independent manipulation of the guiding tool 196 and the electrode (e.g., tail portion 184). For example, the tail portion 184 of the electrode 28 may be manipulated (e.g., bent and/or by a user) to exit the turret frame 36 and/or the turret insert 40 (e.g., from the first channel 88 through the second channel 100 and out of the side opening 104) such that the turret frame and/or the turret insert may rotate independently from the electrode (i.e., a deployed electrode). The guiding tool 196 may also be removed or released or retracted (e.g., by the user) as shown in FIG. 12 from the deployed electrode 28.

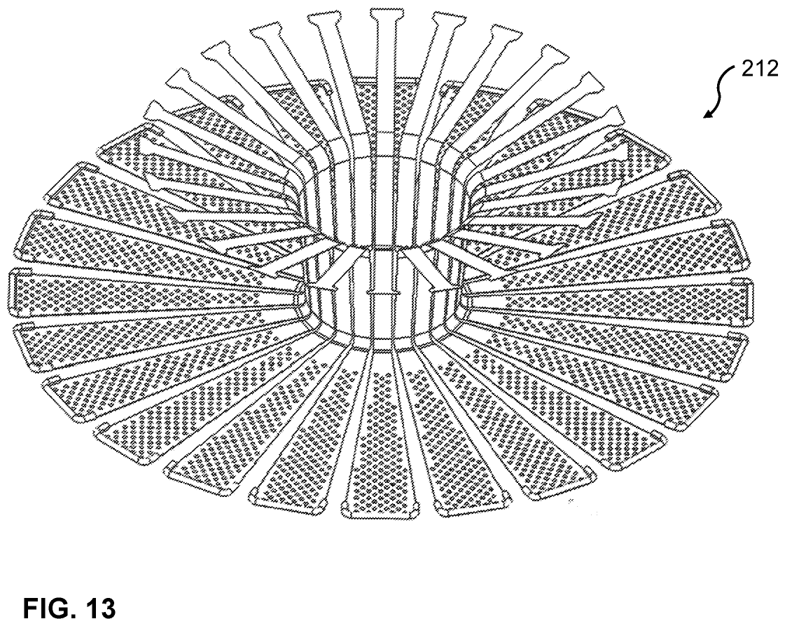

FIG. 13 shows one or more electrodes 212 deployed and without guiding tools, according to some examples. The one or more electrodes 212 may be an electrode array. Once an electrode (e.g., the electrode 28 of FIG. 6) is deployed (e.g., bent with the guiding tool 196 removed as in FIG. 12), the turret frame 36 and/or the turret insert 40 may be rotated (e.g., by the user) independently from the deployed electrode such that the user may deploy another electrode and/or tool through the cortical access system (e.g., the cortical access system 24 of FIG. 2). In other terms, an inserted electrode may be released from the turret frame 36 and turret insert 40 to be deployed. The turret frame 36 and/or the turret insert 40 may include a manipulating member (not shown) configured to help the user rotate the turret frame and/or turret insert.

As shown, the one or more electrodes 212 may be deployed to be substantially near each other, such as having a gap of less than 10 mm, such as less than 5 mm, such as less than 1 mm. The body portion or the head portion (e.g., head portion 180) of each of the one or more electrodes 212 (e.g., electrode 28) may be generally wedge-shaped such that the one or more electrodes 212 may be circumferentially distributed to cover a substantial area within a circle, such as more than 75%, such as more than 85%, such as more than 95% of the area within the circle. The circle may be generally outlined by the distal ends (e.g., distal end 56) and/or the distal bumpers (e.g., bumper 192). Each of the one or more electrodes 212 may be generally wedge-shaped to cover an angle, such as an angle being a fraction of a full circle (i.e. 360°). For example, each of the wedge-shaped electrodes 212 may cover 180° (i.e., with 2 electrodes), 90° (i.e., with 4 electrodes), 60° (i.e., with 6 electrodes), 30° (i.e., with 12 electrodes), 15° (i.e. with 24 electrodes), 10° (i.e. with 36 electrodes), 5° (i.e., with 72 electrodes), or 1° (i.e., with 360 electrodes).

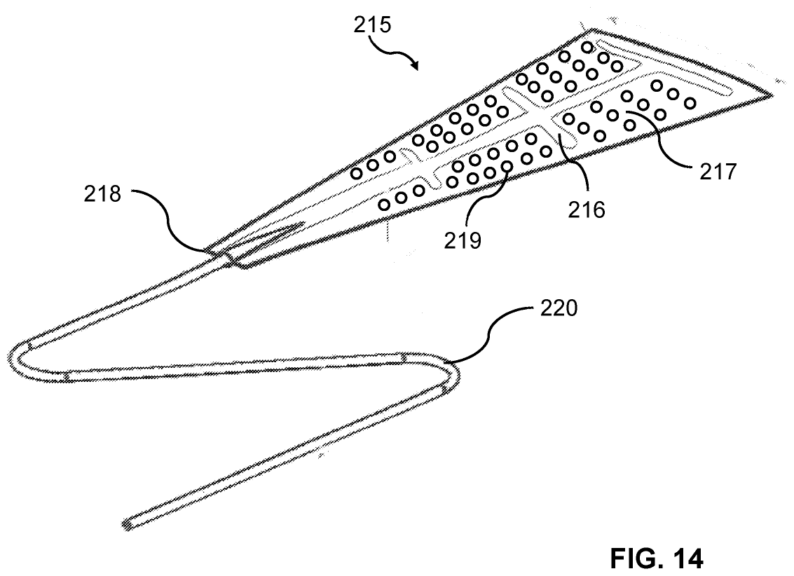

FIG. 14 shows an electrode of the intracranial apparatus 20 of FIG. 1, according to some examples. Additionally or alternatively to what has been described for the electrode 28 of FIG. 6 or one of the one or more electrodes 212 of FIG. 13, an electrode 215 of an intracranial apparatus 20 may include one or more delivery structures such as fluid chambers 216 (or fluid-receiving channels) disposed at least in, on, or at the head portion 217 of the electrode. The fluid chambers 216 are fluidically connected to a fill tube 220 near the proximal end 218 of the electrode. The fill tube 220 is configured to transport fluid (e.g., gas, liquid, solid, gel, suspension, or a mixture of the forgoing) in and out of the fluid chambers 216 to change a fluid quantity in the fluid chambers to transition or actuate at least between an initial state and a positive and/or negative state. For example, fluid quantity may be increased from an initial quantity at the initial state to a first quantity of the positive state such that the shape of the electrode 215 may be altered to obtain improved coupling between the electrode contacts 219 and a patient's brain tissue. Fluid quantity may also be decreased from the first quantity to the initial quantity or further to a second quantity of the negative state such that the electrode 215 is compliant to help its insertion into the patient's skull. Fluid quantity may also be altered to achieve the desired electrode stiffness for improved stability (i.e., ability to stay in place in response to movement of the patient and/or manipulation of the user). The fluid chambers 216 may be configured to collectively and/or independently filled and purged. For example, the fluid chambers 216 may be configured or arranged such that in response to a sequential change in fluid quantities in the chambers, the electrode 215 may move (e.g., crawl, move caterpillar-like, or move snake-like). In various embodiments, the fluid chambers 216 may be filled with fluid such that the rigidity of the electrode is adequate to enable the electrode to be deployed, unfurled, and/or advanced as needed to be positioned in the subdural space.

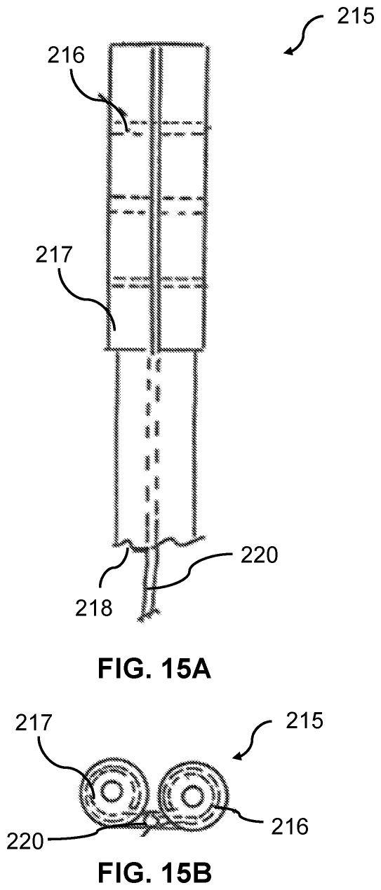

FIG. 15A-15B show the electrode 215 of FIG. 14 in a rolled-up state, viewing from the front (FIG. 15A) and the proximal end (FIG. 15B), according to some examples. The electrode 215 may be biased to take a delivery or rolled-up state, for example, when in the initial state having the initial fluid quantity or when in the negative state having the second fluid quantity. The electrode 215 in the rolled-up state may help its insertion into regions of the brain that are more difficult to access in the deployed or unrolled state (e.g., as in FIG. 14). In the rolled-up state, the width of the electrode 215 may be reduced compared to the planar state. The electrode 215 may transition between the rolled-up state and the unrolled state in response to change in fluid quantity in the fluid chambers 216 (e.g., via the fill tube 220). As shown, at least the head portion 217 of the electrode 215 may be rolled-up in the rolled-up state. The electrode 215 may also roll into a single roll, two rolls (as shown), or more rolls in the rolled-up state. The electrode 215 may include a body (e.g., a body similar to the body portion of electrode 28) movable between the rolled-up state (e.g., delivery state) at which the body has a first width (e.g., width at the distal end in the rolled-up state) and a first length defining a first two-dimensional area, and the unrolled state (e.g., deployed state) at which the body has a second width (e.g., width at the distal end in the planar state) and a second length defining a second two-dimensional area. The second width may be greater than the first width, the second length may be greater than the first length, and/or the second two-dimensional area may be greater than the first two-dimensional area. A length (e.g., a first dimension) may be in the radial direction when inserted into the cortical access system 24 (as in FIG. 9) and a width (e.g., a second dimension) may be in the direction perpendicular to the radial direction. The length of the electrode 215 is greater than its width at least in one of the unrolled and rolled-up state. The electrode (e.g., the body) may be actuated between the delivery state and the deployed state via the delivery structure (e.g., fluid chambers 216), for example, by changing fluid quantities.

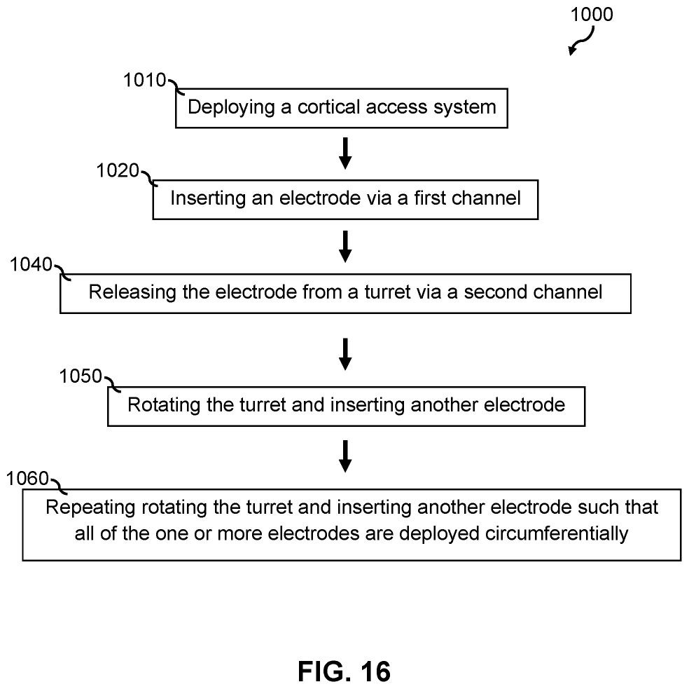

FIG. 16 depicts an illustrative method 1000 for deploying one or more electrodes (e.g., the electrode 28 and/or the one or more electrodes 212) of an intracranial apparatus (e.g., intracranial apparatus 20), according to some examples. The method 1000 includes deploying 1010 a cortical access system (e.g., cortical access system 24) at a patient's cranial opening to access the brain tissue, inserting 1020 an electrode through the patient's cranial opening via a first channel (e.g., first channel 88) onto the patient's brain tissue under the skull, and releasing 1040 the electrode from a turret insert (e.g., turret insert 40) via the second channel (e.g., second channel 100), for example, by removing the tail portion (e.g., tail portion 184) of the electrode from the first channel (e.g. first channel 88) through the second channel and out of the turret insert 40. The method 1000 may further include rotating 1050 the turret insert such that another of the one or more electrodes can be inserted, and inserting 1050 another of the one or more electrodes. The method 1000 may further include repeating 1060 repeating rotating the turret insert (e.g., to align the exit opening 96 of the turret insert at circumferentially spaced locations) and inserting another of the one or more electrodes such that all of the one or more electrodes are deployed circumferentially. The one or more electrodes may be deployed circumferentially (e.g., around the cranial opening) to cover a substantial area within a circle.

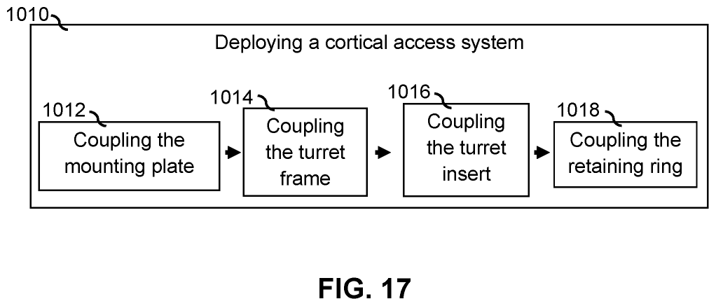

FIG. 17 depicts an illustrative method 1010 for deploying a cortical access system (e.g., cortical access system 24), according to some examples. The method 1010 includes coupling 1012 a mounting plate (e.g., mounting plate 32) to a patient's cranium near a cranial opening via securing elements (e.g., securing elements 48), coupling 1014 a turret frame (e.g., turret frame 36) to the mounting plate, coupling 1016 the turret insert (e.g., turret insert 40) to the turret frame, and coupling 1018 a retaining ring (e.g., retaining ring 44) to the mounting plate such that the turret insert is rotatable.

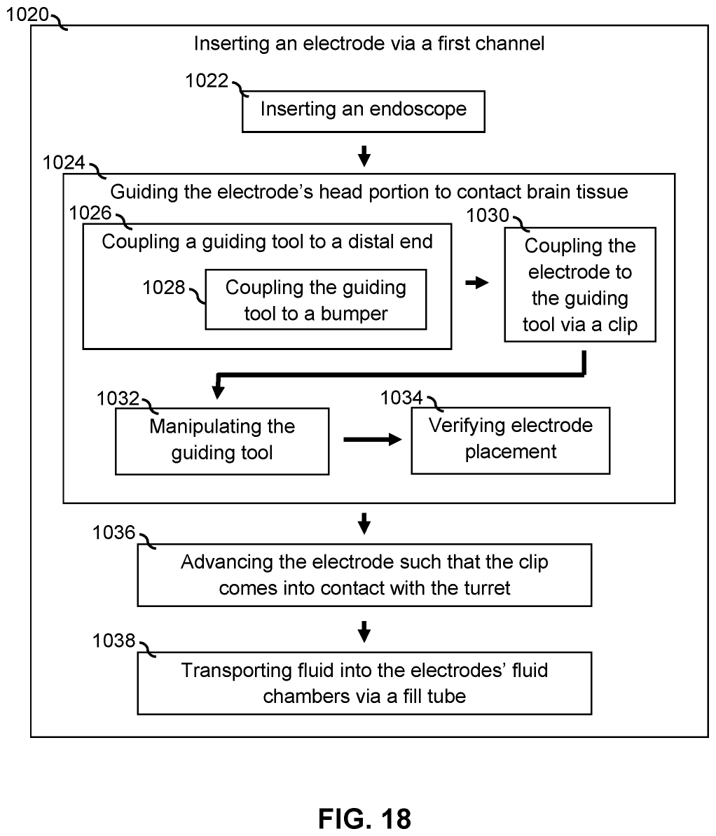

FIG. 18 depicts an illustrative method for inserting 1020 an electrode (e.g., the electrode 28 and/or the one or more electrodes 212) via a first channel (e.g., first channel 88), according to some examples. The method 1020 may include inserting 1022 an endoscope through a patient's cranial opening via a cortical access system (e.g., cortical access system 24). The method 1020 includes guiding 1024 the electrode's head portion (e.g., head portion 180) out of a first channel (e.g., first channel 88) to be near or in contact with the patient's brain tissue. Guiding 1024 the electrode's head portion may include coupling 1026 a guiding tool (e.g., guiding tool 196) to the electrode's distal end (e.g., distal end 176) and manipulating 1032 the guiding tool to guide the electrode. Coupling 1026 the guiding tool may include coupling 1028 the guiding tool to the electrode's bumper (e.g., bumper 192). Guiding 1024 the electrode's head portion may include coupling 1030 the electrode and the guiding tool via a clip (e.g., clip 208) near the proximal end (e.g., at the tail portion 184). Guiding 1024 the electrode's head portion may further include verifying 1034 the placement of the electrode via visualizing the location of the bumper. The method 1020 may further include visualizing the insertion and/or position of the electrode(s) using an imaging instrument (e.g., the endoscope 152) The method 1020 may further include advancing 1036 the electrode such that the clip comes into contact with the turret insert (e.g., turret insert 40). The method 1020 may further include transporting 1038 fluid into the electrode's fluid chambers (e.g., fluid chambers 216) via a fill tube (e.g., fill tube 220) to transition the electrode from an initial state to a positive state.

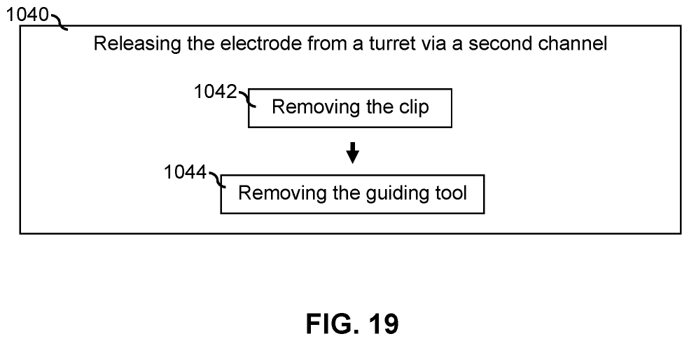

FIG. 19 depicts an illustrative method for releasing 1040 an electrode (e.g., the electrode 28 and/or the one or more electrodes 212) from a turret insert (e.g., turret insert 40) via a second channel (e.g., second channel 100), according to some examples. The electrode may be released from the turret insert such that the turret insert is rotatable independently from the electrode. The method 1040 includes removing 1042 the clip such that the electrode's tail portion (e.g., tail portion 184) is manipulatable independently from a guiding tool (e.g., the guiding tool 196). The method 1040 may further include removing 1044 the guiding tool from the electrode.

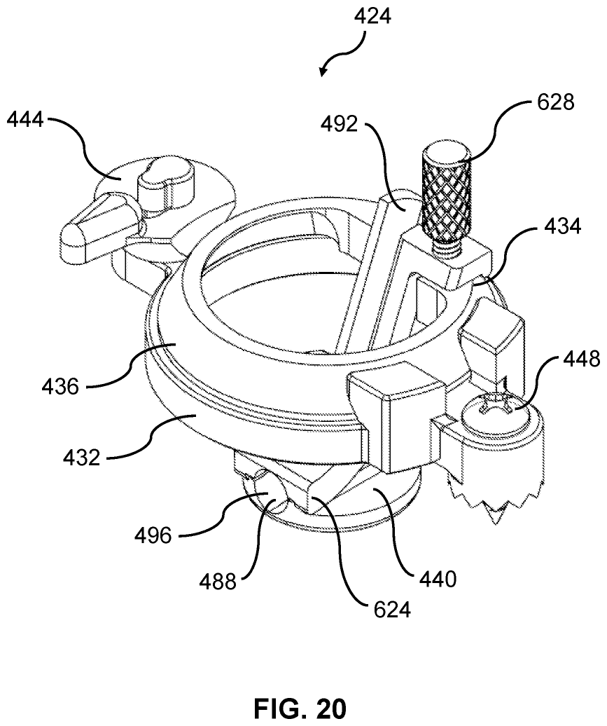

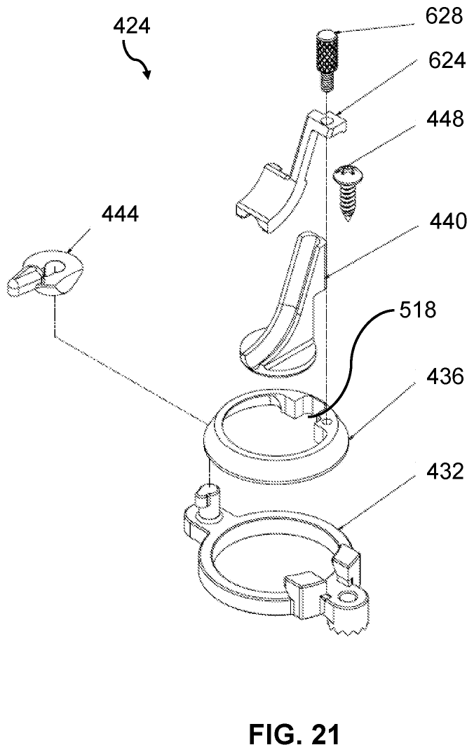

FIG. 20 and FIG. 21 show a cortical access system 424, according to some examples. The diagrams are merely examples, which should not unduly limit the scope of the claims. One of ordinary skill in the art would recognize many variations, alternatives, and modifications. The cortical access system 424 may be similar to the cortical access system 24 and includes similar elements and/or functions. As illustrated, the cortical access system 424 includes a turret base 432, a turret 434 including a turret frame 436, and a turret guide 440, a turret lock 444, and a guide clamp 624. Although the above has been shown using a selected group of components for the system, there can be many alternatives, modifications, and variations. For example, some of the components may be expanded and/or combined. Other components may be inserted to those noted above. Depending upon the embodiment, the arrangement of components may be interchanged with others replaced.

In various examples, the cortical access system 424 is configured to guide a medical tool (e.g., the electrode 28 or the endoscope 152) into a patient's skull (e.g., through the cranial opening 806, for example, as shown in FIG. 8), such as into the patient's subdural space. For example, the cortical access system 424 is configured to be mounted to the patient's skull, particularly to the patient's cranium 802 (e.g., as shown in FIG. 4) to aid the insertion of the medical tool to a target intracranial location, such as on the patient's brain tissue 804. In certain embodiments, the cortical access system 424 is configured to deploy an endoscope 152 and to enable, medical procedures (e.g., evacuation of sub-dural hematoma) to be performed by another medical tool.

In some embodiments, the turret base 432 is configured to be mounted to the patient's skull, such as to be secured to the patient's skull via securing elements 448 (e.g., screws, bolts, clips, and/or O-rings). In various examples, the turret base 432 is configured to receive and/or secure the turret 434, such as to receive and/or secure the turret frame 436. For example, the turret base 432 is configured to receive and secure the turret frame 436 such that the turret 434 can or is able to limit translational motion but allow rotational motion of the turret frame 436 with respect to the turret base 432.

In certain embodiments, the turret frame 436 is configured to receive and/or secure the turret guide 440. For example, the turret frame 436 defines a guide receptacle 518 configured to receive the turret guide 440. In various examples, the turret frame 436 and the turret guide 440, when coupled and/or secured (e.g., via a guide clamp 624 and/or a clamp lock 628), are configured to rotate together.

In various examples, the turret guide 440 defines a guide channel or guide ramp 488 extending from a guide entrance opening 492 to a guide exit opening 496. In certain embodiments, the guide ramp 488 is configured to guide or direct a medical tool (e.g., the electrode 28 or the endoscope 152) or any other tools (e.g., a stereotactic pointer, a suction device, or a biopsy device) or components from outside of the skull to a subdural space inside of the skull (e.g., by entering from the guide entrance opening 492 and extending out from the guide exit opening 496). In some examples, the guide ramp 488 is curved or slanted, such as extending from a top to a side of the cortical access system 424.

In various embodiments, the turret lock 444 is configured to be coupled to the turret base 432 to limit translational motion but allow rotational motion of the turret frame 436 and/or the turret guide 440 (e.g., when coupled to the turret frame 436) with respect to the turret base 432. In certain examples, the turret lock 444 is a cam lock configured to rotate between an open position and a secure position. In various examples, the turret lock 444 is configured to be actuated to a position that prevents rotation of the turret frame 436 with respect to the turret base 432.

In some examples, the guide clamp 624 is configured to be coupled to the turret guide 440 to help secure the turret guide 440 to the turret frame 436, such as via the clamp lock 628. In various examples, the guide clamp 624 is configured to help secure the medical tool (e.g., the electrode 28 or the endoscope 152) to the turret guide 440, such as to the guide ramp 488 of the turret guide 440. In some examples, the clamp lock 628 is configured similarly as the turret lock 444, such as a cam lock configured to rotate between an open position and a secure position.

### Use Case Example

The use example demonstrates the use of a minimally invasive endoscopic assisted device (e.g., intracranial apparatus 20, cortical access system 24, and/or cortical access system 424) for subdural electrode implantation in Epilepsy. The use example is pertinent to subdural grids and strip electrodes which provide wide coverage of the cerebral cortex, precise delineation of the extent of the seizure onset zone, and improved spatial sampling to perform functional mapping for eloquent cortex. The use example describes a novel device which allows for a minimally invasive approach to implantation of subdural grid and strip electrodes.

In the use case, a skull mounted device is configured to allow for implantation of subdural electrodes through a keyhole craniotomy with direct visualization using the aid of a flexible neurovideoscope. The initial studies in preparation for grid development performed on cadaveric skulls were analyzed to determine the size of craniotomy required for deployment, maximal distance of strip electrode deployment from center of craniotomy, and visual inspection of the cortex was performed for any underlying damage.

The device allowed for the placement of subdural electrodes through a 40 mm craniotomy. Subdural electrodes were deployed in multiple directions to a distance of a 70 mm radius from the center of the craniotomy. There was no visual damage to the underlying cortex after the procedures were completed.

Large craniotomies are typically desired to provide direct visualization of the implantation of subdural electrodes, but can increase the risk of subdural hemorrhages and infections. This use case describes a novel minimally invasive endoscopically assisted device for the implantation of subdural strip electrodes under direct visualization. The use case shows this device is capable for limiting the size of the craniotomy, avoiding incision through the temporalis muscle, and implanting subdural electrodes with visualization of the cortex.

The device combines the benefits of open surgery with those of an endoscopic approach for grid placement. In response, an electrode surgical delivery device was devised capable of enabling endoscopic operative imaging and improved electrode delivery. The device provides the ability to insert an endoscope into the subdural space to visualize navigation along the surface of the cerebral cortex. In addition, similar size electrode arrays as used for open surgery can be delivered through the device. With this platform, the device can be configured for deployable cortical coverage. The application of this device greatly reduced craniotomy size compared to the traditional approach and has the potential to access the majority of the cortical convexity. Given the limitations of current surgical procedure, embodiments of the invention provide a novel minimally invasive endoscopic assisted device for the placement of subdural electrodes in subdural grid electroencephalography (sdEEG).

The device includes a mounting plate, a turret, an electrode holder, and a retaining ring. The mounting plate is used to affix the delivery device to the skull. The turret is used to retain the endoscope and the electrode array. The retaining ring is used to hold the turret in place with the appropriate force and provide the ability to rotate the delivery device within the mounting plate to change imaging and electrode delivery direction.

This device was tested on three cadaveric heads, bilaterally, for a total of six trials of the device. After fixation in a head-holder, a linear incision was made, being careful to avoid the termporalis muscle. The dura was then opened in a cruciate manner and tacked back with sutures. Following this, the cranial plate was mounted and the turret was locked in place with the locking ring. Once this was completed, a Storz flexible videoneuroscope was introduced through the scope channel and the subdural space was navigated. Following this a standard four contact sdEEG strip electrode was deployed using the working channel of the turret. The turret was then removed and the underlying cortex was visually examined. Measured variables included the size of craniotomy required for deployment, maximal distance of electrode deployment from center of craniotomy, and the quality of the underlying cortex once the electrode deployment had been completed.

In embodiments, the device comprises a turret with a separate channel for a flexible endoscope, a locking ring, and a cranial mounting plate. The overall width of the device when mounted to the skull is 61 mm. The opening in the mounting plate for the turret is 45 mm. When the turret is depressed into the mounting plate the plunge depth is 22.23 mm below the outer cortex of the skull. The average bone thickness in the frontal area ranges from 6-8 mm; the device extends 1.4-1.6 cm below the inner table however this can be adjusted. The diameter of the turret itself is 38 mm, which requires a 40 mm craniotomy for adequate placement. A standard 4-contact (1 cm spacing) sdEEG strip electrode can be deployed to approximately 8 cm from the turret to the distal contact with endoscopic visualization. With the turret removed, the cortex was visually inspected for any injury from the device, and no obvious injury was noted. The device did not leave an impression on the cortex.

In this use study, the device is configured to allow hybrid electrophysiologic recordings with both sdEEG and stereoencephalography (sEEG). After implantation of sEEG depth electrodes, a linear incision would be created in an area devoid of sEEG electrodes. A small craniotomy would then be fashioned, and subdural strip electrodes would be placed on cortical areas of interest under direct visualization. One significant benefit of this approach would be the direct visualization of bridging veins and the potential ability to either avoid them or control them using instruments through the endoscope. Additionally, as the turret can rotate 360 degrees, the device can be used with a circular subdural strip electrode array that covers a wide area of cortex, as we can place electrodes approximately 7 cm from the device.

This use case describes a concept study describing a novel minimally invasive surgical approach introducing an endoscopically assisted device for the implantation of subdural strip electrodes under direct visualization. Embodiments involve creating the device using biocompatible materials that would allow for sterilization and reprocessing, and using this device in a population of patients undergoing intracranial electroencephalography (iEEG) for epilepsy.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. A cortical access system for delivery of one or more electrodes into an epidural and/or sub dural space and onto a patient's brain tissue through a cranium opening, the system comprising:
a turret including a proximal end portion, a distal end portion, and a first channel extending from an entrance opening to an exit opening, the first channel configured to guide the one or more electrodes from the entrance opening to the exit opening for positioning on the patient's brain tissue.

2. The cortical access system of claim 1, wherein:
the distal end portion includes a periphery; and
the exit opening of the first channel is on the periphery of the distal end portion, and the channel is configured to guide the one or more electrodes onto the patient's brain tissue below the cranium.

3. The cortical access system of claim 1, wherein the turret further comprises a second channel configured for the one or more electrodes to be released from the turret.

4. The cortical access system of claim 3, wherein the second channel is narrower than the first channel such that only a portion of each of the one or more electrodes is releasable via the second channel.

5. The cortical access system of claim 1, wherein the first channel is curved or sloped to form a ramp for guiding the one or more electrodes out of the exit opening generally horizontally between the patient's brain tissue and cranium.

6. The cortical access system of claim 1, wherein the turret comprises a turret frame and a turret insert removably mated to the turret frame, and wherein the turret insert defines the first channel.

7. The cortical access system of claim 1, further comprising a mounting plate configured to be secured to the patient's cranium at the cranial opening via securing elements and to rotatably receive the turret.

8. The cortical access system of claim 7, further comprising a retaining ring configured to be coupled to the mounting plate such that the turret is rotatable while translational motion of the turret is limited.

9. The cortical system of claim 1, wherein the turret is adjustable for different skull thicknesses.

10. The cortical access system of claim 1, wherein the turret further comprises an endoscope channel configured to receive an endoscope for imaging of an electrode being delivered by the system.

11. The cortical access system of claim 1, wherein the turret includes a bottom surface configured to atraumatically displace brain tissue and increase sub-dural space.

12. The cortical access system of claim 1, wherein the turret further comprises a force sensor configured to monitor a force exerted onto the patient's brain tissue.

13. An electrode, comprising:
a head having a proximal end portion and a distal end portion wider than the proximal end portion;
a tail extending from the proximal end portion of the head; and
one or more electrode contacts disposed on the head;
wherein the electrode is flexible and configured to be inserted through a cortical access system to access a patient's brain tissue.

14. The electrode of claim 13, further comprising a bumper at the distal end portion of the head.

15. The electrode of claim 14, wherein the bumper is radiopaque.

16. The electrode of claim 14, wherein the bumper is configured to releasably receive a guiding tool.

17. The electrode of claim 16, wherein manipulation of the guiding tool causes movement of the electrode.

18. The electrode of claim 16, wherein the tail is configured to be releasably coupled to the guiding tool via a clamp.

19. The electrode of claim 13, further comprising a tab at a proximal end of the tail.

20. The electrode of claim 13, wherein the electrode comprises a biocompatible dielectric substrate, a conductive layer coupled to the substrate, and a biocompatible dielectric top layer coupled to the conductive layer.

21. The electrode of claim 20, wherein the biocompatible dielectric substrate and/or the biocompatible dielectric top layer comprises at least one of polyimide, Parylene-C, and silicone.

22. The electrode of claim 20, wherein the conductive layer comprises at least one of platinum, titanium-platinum, gold, copper, and tin.

23. The electrode of claim 13, wherein the head is configured to be movable through a first channel of the cortical access system and not movable through a second channel of the cortical access system.

24. The electrode of claim 13, wherein the tail is configured to be movable through the second channel of the cortical access system.

25. The electrode of claim 13, wherein the tail is configured to be manipulated such that the electrode is releasable from a turret of the cortical access system to allow another electrode to be inserted through the cortical access system.

26. The electrode of claim 13 is generally wedge-shaped such that a plurality of the electrodes is circumferentially distributable on the patient's brain tissue.

27. The electrode of claim 13, further comprising one or more fluid chambers disposed at least at the head.

28. The electrode of claim 27, wherein each fluid chamber is fluidically connected to a fill tube at a proximal end of the electrode.

29. The electrode of claim 28, wherein the fill tube is configured to transport fluid in and out of each fluid chamber to change fluid quantities in the fluid chamber.

30. The electrode of claim 29, wherein the one or more fluid chambers are configured to transition the electrode between an initial state and a positive state in response to a change in fluid quantity in the one or more fluid chambers.

31. The electrode of claim 29, wherein the electrode has a variable stiffness corresponding to a fluid quantity in the one or more fluid chambers.

32. The electrode of claim 29, wherein the electrode is configured to move in response to a sequential change in fluid quantities in the one or more chambers.

33. The electrode of claim 29, wherein the electrode is configured to transition between a delivery state at which the electrode has a first width, and a deployed state at which the electrode has a second width greater than the first width, in response to change in fluid quantities in the one or more chambers.

34. A method for deploying one or more electrodes of an intracranial apparatus including a cortical access system having a turret with a first channel, comprising:
deploying the cortical access system at a patient's cranial opening;
inserting an electrode through the patient's cranial opening via the first channel to access the patient's brain tissue; and
releasing the electrode from the turret such that the turret is rotatable independently of the released electrode.

35. The method of claim 34, wherein the cortical access system further includes a mounting plate and a retaining ring, wherein deploying the cortical access system comprises:
coupling the mounting plate to a patient's cranium at the cranial opening via securing elements;
coupling the turret to the mounting plate; and
coupling the retaining ring to the mounting plate such that the turret is rotatable.

36. The method of claim 35, wherein the turret includes a turret frame and a turret insert, and wherein coupling the turret to the mounting plate includes:
coupling the turret frame to the mounting plate; and
coupling the turret insert to the turret frame.

37. The method of claim 34, wherein inserting the electrode comprises guiding the electrode's head out of the first channel to a location between the patient's cranium and brain tissue.

38. The method of claim 37, wherein guiding the electrode's head comprises:
coupling a guiding tool to the electrode head; and
manipulating the guiding tool to guide the electrode.

39. The method of claim 38, wherein coupling the guiding tool comprising coupling the guiding tool to a bumper on the electrode's head.

40. The method of claim 39, wherein guiding the electrode's head further comprises verifying the placement of the electrode via visualizing the location of the bumper.

41. The method of claim 38, wherein guiding the electrode's head further comprising coupling the electrode and the guiding tool via a clip.

42. The method of claim 41, wherein inserting the electrode comprises advancing the electrode such that the clip comes into contact with the turret.

43. The method of claim 42, wherein releasing the electrode comprises removing the clip such that the electrode's tail is manipulatable independently from the guiding tool.

44. The method of claim 38, wherein releasing the electrode comprises removing the guiding tool from the electrode.

45. The method of claim 34, wherein the electrode is configured to be released such that the turret is rotatable independently from the electrode.

46. The method of claim 34, further comprising:
rotating the turret such that another of the one or more electrodes can be inserted; and
inserting another of the one or more electrodes.

47. The method of claim 46, further comprising repeating rotating the turret and inserting another of the one or more electrodes such that the one or more electrodes are deployed circumferentially.

48. The method of claim 47, wherein the one or more electrodes are deployed circumferentially to cover a substantial area within a circle.

49. The method of claim 34, further comprising inserting an endoscope through the patient's cranial opening via the cortical access system and using the endoscope to visualize the electrode.

50. The method of claim 34, wherein releasing the electrode from the turret comprises releasing the electrode through a second channel in the turret that is connected to the first channel.

51. A cortical access system for delivering a medical tool into an epidural and/or subdural space and onto a patient's brain tissue through a cranium opening, the system comprising:
a turret including a proximal end portion, a distal end portion, and a first channel extending from an entrance opening to an exit opening, the first channel configured to guide the medical tool from the entrance opening to the exit opening for positioning on the patient's brain tissue.

52. The cortical access system of claim 51, wherein:
the distal end portion includes a periphery; and
the exit opening of the first channel is on the periphery of the distal end portion, and the channel is configured to guide the medical tool onto the patient's brain tissue below the cranium.

53. The cortical access system of claim 51, wherein the first channel is curved or sloped to form a ramp for guiding the medical tool out of the exit opening generally horizontally between the patient's brain tissue and cranium.

54. The cortical access system of claim 51, wherein the turret comprises a turret frame and a turret guide removably mated to the turret frame, and wherein the turret guide defines the first channel.

55. The cortical access system of claim 51, further comprising a turret base configured to be secured to the patient's cranium at the cranial opening via a securing means and to rotatably receive the turret.

56. The cortical access system of claim 55, further comprising a turret lock configured to be coupled to the turret base such that the turret is rotatable while translational motion of the turret is limited.

57. The cortical access system of claim 51, wherein the turret includes a bottom surface configured to atraumatically displace brain tissue and increase sub-dural space.

58. The cortical access system of claim 51, further comprising a guide clamp configured to be coupled to the turret to help secure the medical tool received in the first channel.

59. The cortical access system of claim 58, further comprising a clamp lock configured to secure the guide clamp to the turret guide.

60. The cortical access system of claim 51, wherein the medical tool is an electrode or an endoscope.
